(19) United States
(12) Patent Application Publication
(10) Pub. No.: US 2004/0087662 A1
(43) Pub. Date: May 6, 2004

(54) Use of accelerated lymphocyte homing agents for the manufacture of a medicament for the treatment of delayed graft function

(76) Inventors: Marc Bigaud, Sundhoffen (FR); Volker Brinkmann, Freiburg (DE); Tomasz Sablinski, Basel (CH)

Correspondence Address: THOMAS HOXIE, NOVARTIS, CORPORATE INTELLECTUAL PROPERTY, ONE HEALTH PLAZA 430/2, EAST HANOVER, NJ, 07936-1080, US

(21) Appl. No.: 10/468,471

(22) PCT Filed: Feb. 21, 2002
(86) PCT No.: PCT/EP02/01860

(30) Foreign Application Priority Data
Feb. 22, 2001 (GB) 01104438.7
Feb. 28, 2001 (GB) 0105000.4

(51) Int. Cl. A61K 31/13

(52) U.S. Cl. 514/669

(57) ABSTRACT

The invention relates to the use of an accelerated lymphocyte homing agent, e.g. a 2-amino-1,3-propanediol derivative, in reducing delayed graft function in a recipient of organ or tissue transplant.

The present invention relates to a new use for an accelerated lymphocyte homing (“ALH”) agent e.g. a 2-amino-1,3-propanediol derivative.

The ALH agents of the invention are compounds which sequester lymphocytes from peripheral tissues to secondary lymphatic organs. Suitable ALH agents include e.g. analogs from myriocin or ISP-1, a natural metabolite of the ascomycetes Isaria sinclairii. Suitable ALH are e.g. 2-aminopropane-1,3-diol compounds of formula I:

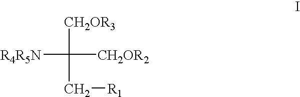

wherein

R₁ is an optionally substituted straight- or branched carbon chain having 12 to 22 carbon atoms which may be optionally interrupted by an optionally substituted phenylene, and each of R₂, R₃, R₄ and R₅, independently, is H or lower alkyl, in free form or in pharmaceutically acceptable salt form.

When the carbon chain as R₁ is substituted, it is preferably substituted by halogen, nitro, amino, hydroxy or carboxy. When the carbon chain is interrupted by an optionally substituted phenylene, the carbon chain is preferably unsubstituted. When the phenylene moiety is substituted, it is preferably substituted by halogen, nitro, amino, methoxy, hydroxy or carboxy.

Preferred compounds of formula I are those wherein R₁ is a straight or branched, preferably straight, chain alkyl having 13 to 20 carbon atoms, optionally substituted by nitro, halogen, amino, hydroxy or carboxy, and, more preferably those wherein R₁ is phenylalkyl substituted by a straight or branched C₆₋₁₄-alkyl chain optionally substituted by halogen and the alkyl moiety is a C₁₋₆alkyl optionally substituted by hydroxy. More preferably, R₁ is phenyl-C₁₋₆alkyl substituted on the phenyl by a straight or branched, preferably straight, C₆₋₁₄alkyl chain. The C₆₋₁₄alkyl chain may be in ortho, meta or para, preferably in para.

Preferably each of R₂ to R₅ is H.

When the compounds of formula I have on or more asymmetric centers in the molecul, th present invention is to be understood as embracing th various optical isomers, as well as racemates, diastereoisomers and mixtures thereof.

Examples of the pharmaceutically acceptable salts of the compounds of formula I include salts with inorganic acids, such as hydrochloride, hydrobromide and sulfate, salts with organic acids, such as acetate, fumarate, maleate, benzoate, citrate, malate, methanesulfonate and benzenesulfonate salts, and when a carboxy group is present, salts with metals such as sodium, potassium, calcium and aluminium, salts with amines, such as triethylamine and salts with dibasic amino acids, such as lysine. Compounds of formula I and salts of the present invention encompass hydrate and solvate forms.

A preferred compound of formula I is 2-amino-2-tetradecyl-1,3-propanediol. A particularly preferred ALH agent for use in the invention is FTY720, i.e. 2-amino-2-[2-(4-octylphenyl) ethyl]propane-1,3-diol, in free form or in pharmaceutically acceptable salt form (hereinafter referred to as Compound A), e.g. the hydrochloride salt, as shown:

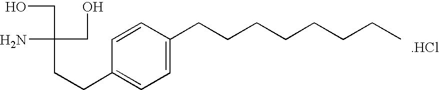

Compounds of formula I have, on the basis of observed activity, e.g. as described in EP-A1-627,406 been found to be useful e.g. as immunosuppressants, e.g. in the treatment of acute allograft rejection or autoimmune disorders.

In accordance with the present invention, it has now surprisingly been found that a ALH agent, e.g. Compound A in free form or in pharmaceutically acceptable salt form, reduces delayed graft function.

Following renal transplantation, approximately 25% of patients exhibit a delay in the onset of renal function in most instances requiring e,g, intervention with hemodialysis; this condition is referred as delayed graft function (DGF). This incidence has remained stable since 1990, despite the introduction of a number of new immunosuppressive drugs in the last decade. The major risk factors that are associated with an increased incidence of DGF are prolonged cold ischemia time and procurement of kidneys from older donors or cadaveric kidneys.

DGF is a dramatic consequence of prolonged cold preservation of an organ prior to its transplantation into a recipient. It is characterized by an impaired functional recovery of th grafts upon rep rfusion and often results in an increased ne d of post-operative care. In case of h art transplantation, DGF may result in death, t mporary heart failure and/or in a ne d for chronotropic (external pacing) or inotropic (pharmacologic agents) supports. In cas of kidney transplantation, DGF may result in temporary kidney failure requiring pharmacological treatments and/or additional dialysis. The severity of DGF is proportional to the duration of cold preservation. Any treatment reducing DGF will not only improve the functional recovery of all grafts subjected to cold preservation but also will reduce the number of grafts that cannot be used because of too long preservation time (so called non-optimal grafts). Ultimately, this may increase the number of grafts available.

Immunosuppressive management of patients with DGF still remains a major unmet medical need in transplantation.

In accordance with the particular findings of the present invention, there is provided:

1.1. A method of reducing DGF in a recipient of organ or tissue transplant comprising administering to said recipient a therapeutically effective amount of an ALH agent, e.g. Compound A in free form or in pharmaceutically acceptable salt form;

1.2 A method of improving functional recovery of a transplanted organ or tissue in a recipient of organ or tissue transplant comprising administering to said recipient a therapeutically effective amount of a ALH agent, e.g. Compound A in free form or in pharmaceutically acceptable salt form.

Organ or tissue transplants include e.g. heart, lung, combined heart-lung, liver, kidney, spleen, small bowell, pancreatic (complete or partial, e.g. Langerhans islets) grafts, bone marrow or stem cells.

1.3 A method of improving glomerular filtration rate of a transplanted kidney in a recipient of kidney transplant comprising administering to said recipient a therapeutically effective amount of a ALH agent, e.g. Compound A in free form or in pharmaceutically acceptable salt form.

1.4 A method as defined above, comprising co-administration of a therapeutically effective amount of an ALH agent, e.g. Compound A in free form or in pharmaceutically acceptable salt form, and a second drug substance, said second drug substance being an immunosuppressant or immunomodulatory drug.

1.5 A method as defined above, comprising co-administration of a therapeutically effective amount of an ALH ag nt, e.g. C mpound A in free form or in pharmaceutically acceptabl salt form, and a s cond drug substance, said second drug substance being an immunosuppressant or immunomodulatory drug other than a calcineurin inhibitor.

Suitable second drug substances may include e.g. a calcineurin inhibitor, e.g. cyclosporin A or FK-506; a macrocyclic lactone which exhibits immunosuppressant properties, e.g. rapamycin or a derivative thereof, e.g. 40-O-(2-hydroxyethyl)rapamycin or 40-[3-hydroxy-2-(hydroxymethyl)-2-methylpropanoate]-rapamycin; corticosteroids; cyclophosphamide; azathioprine; methotrexate; brequinar; leflunomide; mizoribine; mycophenolic acid; mycophenolate mofetil; 15-deoxyspergualine or a derivative thereof; immuno-suppressive monoclonal antibodies, e.g., monoclonal antibodies to leukocyte receptors, e.g., to MHC, CD2, CD3, CD4, CD7, CD11a/CD18, CD25, CD28, B7, CD40, CD45, CD58, CD137, ICOS, CD150, OX40, 4-1BB or to their ligands; or other immunomodulatory compounds, e.g. CTLA4-Ig or an analog, homolog or derivative thereof, e.g. LEA29Y.

The terms “co-administration” or “combined administration” or the like as utilized herein are meant to encompass administration of the selected therapeutic agents to a single patient, and are intended to include treatment regimens in which the agents are not necessarily administered by the same route of administration or at the same time.

2. An ALH agent, e.g. Compound A in free form or in pharmaceutically acceptable salt form, for use in any method as defined under 1.1 to 1.5 above; or

3. An ALH agent, e.g. Compound A in free form or in pharmaceutically acceptable salt form, for use in the preparation of a pharmaceutical composition for use in any method as defined under 1.1 to 1.5 above; or

4. A pharmaceutical composition for use in any method as defined under 1.1 to 1.5 above comprising an ALH agent, e.g. Compound A in free form or in pharmaceutically acceptable salt form, together with one or more pharmaceutically acceptable diluents or carriers therefor.

5. A pharmaceutical combination comprising:

a) a first agent which is an ALH agent, e.g. Compound A in free form or in pharmaceutically acceptable salt form, and

b) a co-agent which is an immunosuppressant or immunomodulatory drug, e.g. as disclosed above,

e.g. for use in any m thod as defined under 1.1 to 1.5 above. Preferably, the co-agent b) is an immunosuppressant or immunomodulatory drug other than a calcineurin inhibitor, e.g. as disclosed above.

The term “pharmaceutical combination” as used herein means a product that results from the mixing or combining of more than one active ingredient and includes both fixed and non-fixed combinations of the active ingredients. The term “fixed combination” means that the active ingredients, e.g. Compound A and a co-agent, are both administered to a patient simultaneously in the form of a single entity or dosage. The term “non-fixed combination” means that the active ingredients, e.g. Compound A and a co-agent, are both administered to a patient as separate entities either simultaneously, concurrently or sequentially with no specific time limits, wherein such administration provides therapeutically effective levels of the agents in the body of the patient. By “co-agent” is meant one or more compounds as disclosed above.

Preferably the ALH agent e.g. Compound A in free form or in pharmaceutically acceptable salt form, is co-administered with an immunosuppressant or immunomodulatory drug, e.g. a calcineurin inhibitor, e.g. cyclosporin A, or a macrocyclic lactone which exhibits immunosuppressant properties, e.g. rapamycin or a derivative thereof.

A preferred combination according to the invention comprises Compound A in free form or in pharmaceutically acceptable salt form, e.g. hydrochloride salt, and, as co-agent b), either rapamycin or a derivative thereof, e.g. 40-O-(2-hydroxyethyl)rapamycin, optionally together with a corticosteroid.

Utility of the ALH agents, e.g. Compound A in free form or in pharmaceutically acceptable salt form, e.g. in the reduction of DGF or improvement of the functional graft recovery, as hereinabove specified, may be demonstrated in animal test methods as well as in clinic, for example in accordance with the methods hereinafter described.

A. In vivo

Kidneys from DA rats are preserved 40 h at 4° C. prior to transplantation and then transplanted as life supporting allografts in Lewis rats. DGF is assessed during 1 week post transplantation by monitoring glomerular filtration rate (GFR). Recipients are treated with the ALH agent to be tested, either alone, or combined with a macrocyclic lactone, e.g. 40-O-(2-hydroxyethyl)rapamycin, or a calcin urin inhibitor. e.g. cyclosporin A in a microemulsion. In th control groups, cyclosporin A is given alone at 5 mg/kg/day, a dose which increases graft survival to at least 80 days. In this assay, the ALH agent whether administered alon or in combination with a calcineurin inhibitor or a macrocyclic lacton improves DGF over the controls.

More particularly, in control animals GFR is reduced by about 65% at 1 week post transplantation, whereas in recipients treated with 0.3 mg/kg/d Compound A hydrochloride+0.625 mg/kg/d 40-O-(2-hydroxyethyl)-rapamycin the reduction of GFR is only 35% at 1 week post transplantation.

B. Clinical Trial

30 to 60 de novo adult renal transplant recipients at increased risk of DGF are included in a one year open-label study. Baseline assessment occurs within 24 hours pre-transplantation. A core renal allograft biopsy is performed prior to implantation and revascularization on Day 0, to serve as a Baseline comparison for evaluation of subsequent biopsies should they be required. Each patient must meet inclusion criteria and none of the exclusion criteria, at Baseline (pre-transplantation) and immediately prior to enrollment in order to participate in this study.

Patients receive a first dose of ALH agent, e.g. Compound A in free form or a pharmaceutically acceptable salt thereof, e.g. orally, at least 2 hours prior to the renal allograft revascularization (Day 0). Maintenance immunosuppression with the ALH agent commences on the morning following transplantation (D 1) The once daily dose is then adjusted for each patient as required.

Patients are treated with their first dose of 40-O-(2-hydroxyethyl)-rapamycin at least 2 hours prior to renal allograft revascularization (Day 0), concurrently with the ALH agent dose. All patients then receive daily an ALH agent dose, e.g. 2.5 mg Compound A hydrochloride, and 1.5 mg 40-(2-hydroxyethyl)-rapamycin bid, the doses being adjusted as required. All patients receive corticosteroids perioperatively and continuing daily for the entire duration of the one-year study. During the one-year study period, patient visits occur on Days 0, 1,7, 14, 28 and at Months 2, 3, 6, 9 and 12. An interim analysis of efficacy and safety will be performed when all patients have completed 3 months on study, with a final analysis at 12 months post-transplantation. The renal function is evaluated by serum creatinine measurements and incidence of proteinuria within 3 and 12 months in patients. A beneficial effect is observed.

Daily dosages required in practicing the method of the present invention will vary depending upon, for example, the ALH agent employed, the host, the mode of administration, the severity of the condition to b treated, and the optionally concomitantly us d immunosuppressive drug .g. rapamycin or a derivativ th r of. A preferred daily dosag range is about from 0.03 to 2.5 mg/kg per day as a single dos or in divided doses. Suitabl daily dosages for patients are on the order of from e.g. 0.5 to 50 mg p.o. Suitable unit dosage forms for oral administration comprise from ca. 0.1 to 25 mg active ingredient, e.g. Compound A, e.g. in hydrochloride form, together with one or more pharmaceutically acceptable diluents or carriers therefor. As an alternative, the ALH agent may also be administered twice or three times a week, e.g. at a dosage as indicated above.

The ALH agents may be administered by any conventional route, in particular enterally, e.g. orally, for example in the form of solutions for drinking, tablets or capsules or parenterally, for example in the form of injectable solutions or suspensions. Pharmaceutical compositions comprising a compound of formula I may be manufactured in conventional manner, e.g. as described in EP-A1-627,406.

Daily dosages with respect to the co-agent used will vary depending upon, for example, the compound employed, the host, the mode of administration and the severity of the condition to be treated. A preferred daily dosage range is about from 0.25 to 25 mg of macrocyclic lactone as a single dose or in divided doses. Suitable daily dosages for patients are on the order of from e.g. 0.2 to 25 mg p.o. rapamycin or 40-O-(2-hydroxyethyl)rapamycin, preferably 0.75 to 5 mg per day. The co-agent b) may be administered by any conventional route, in particular enterally, e.g. orally, e.g. In the form of tablets, capsules, drink solutions, nasally, pulmonary (by inhalation) or parenterally, e.g. in the form of injectable solutions or suspensions. Suitable unit dosage forms for oral administration comprise from ca. 0.05 to 12.5 mg rapamycin or 40-O-(2-hydroxyethyl)-rapamycin, together with one or more pharmaceutically acceptable diluents or carriers therefor.

Compounds of formula I in free from or in pharmaceutically acceptable salt form are well tolerated at dosages required for use in accordance with the present invention. For example, the acute LD₅₀ is >10 mg/kg p.o. in rats and monkeys.

1. Use of an accelerated lymphocyte homing agent, in free form or in pharmaceutically acceptable salt form in the preparation of a pharmaceutical composition for use in reducing delayed graft function in a recipient of organ or tissue transplant.

2. Use of an accelerated lymphocyte homing agent, in free form or in pharmaceutically acceptable salt form, according to claim 1 for use in improving functional recovery of a transplanted organ or tissue in a recipient of organ or tissue transplant.

3. Use of an accelerated lymphocyte homing agent, in free form or in pharmaceutically acceptable salt form, according to claim 1 for use in improving glomerular filtration rate of a transplanted kidney in a recipient of kidney transplant.

4. Use according to claim 1 wherein the accelerated lymphocyte homing agent is a compound of formula I 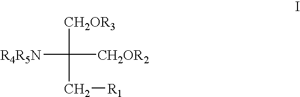
wherein
R₁ is an optionally substituted straight- or branched carbon chain having 12 to 22 carbon atoms which may be optionally interrupted by an optionally substituted phenylene, and each of R₂, R₃, R₄ and R₅, independently, is H or lower alkyl, in free form or in pharmaceutically acceptable salt form.

5. Use according to claim 4, wherein the compound of formula I is 2-amino-2-[2-(4-octylphenyl) ethyl]propane-1,3-diol, in free form or in pharmaceutically acceptable salt form.

6. A pharmaceutical composition for use in reducing delayed graft function or improving functional recovery of a transplanted organ or tissue in a recipient of organ or tissue transplant, or in improving glomerular filtration rate of a transplanted kidney in a recipient of kidney transplant comprising an accelerated lymphocyte homing agent, in free form or in pharmaceutically acceptable salt form, together with one or more pharmaceutically acceptabl dilu nts or carriers therefor.

7. A pharmaceutical combination for use in reducing delay d graft function or improving functional recovery of a transplanted organ or tissue in a recipient of organ or tissue transplant or improving glomerular filtration rate of a transplanted kidney in a recipient of kidney transplant comprising
a) a first agent which is an accelerated lymphocyte homing agent, in free form or in pharmaceutically acceptable salt form, and
b) a co-agent which is an immunosuppressant or immunomodulatory drug.

8. A combination according to claim 7 wherein the co-agent is other than a calcineurin inhibitor.

9. A composition according to claim 6 or a combination according to claim 7 wherein the accelerated lymphocyte homing agent is a compound of formula I as stated in claim 4.

10. A method of reducing delayed graft function or improving functional recovery of a transplanted organ or tissue in a recipient of organ or tissue transplant or improving glomerular filtration rate of a transplanted kidney in a recipient of kidney transplant comprising administering to said recipient a therapeutically effective amount of an accelerated lymphocyte homing agent, in free form or in pharmaceutically acceptable salt form.
